(19) United States
(12) Plant Patent Application Publication
(10) Pub. No.: US 2017/0374778 P1
(43) Pub. Date: Dec. 28, 2017

(54) Hydrangea plant named `BCHY-11.026'.

(50) ;

(71) Applicant: Harrison M. Higaki, San Mateo, CA (US)

(72) Inventor: Harrison M. Higaki, San Mateo, CA (US)

(21) Appl. No.: 14/999,797

(22) Filed: Jun. 27, 2016

(51) Int. Cl. A01H 5/02

(52) U.S. Cl. CPC A01H 5/02
USPC PLT/250

(57) ABSTRACT

A new and distinct cultivar of Hydrangea macrophylla (Thunb.) named ‘BCHY-11.026’ originated as a controlled cross between varieties. The variety ‘BCHY-11.026’ has attractive inflorescences with relatively large sepalous florets, attractive, nonuniform sepal pigmentation and good commercial characteristics. Sepalous florets ring the non-sepalous florets in the center of the inforescence.

Botanical classification: Hydrangea macrophylla (Thunb.) ‘BCHY-11.026’.

Variety denomination: ‘BCHY-11.026’.

### BACKGROUND OF THE INVENTION

This invention relates to a new and distinct cultivar of the Saxifragaceae family. The botanical name of the plant is Hydrangea macrophylla (Thunb.) ‘BCHY-11.026’.

The new cultivar originated as a seedling from a controlled cross between the unpatented variety known as ‘8.1’ which was the seed parent and the variety ‘BC8.3’ which was the pollen parent. The pollen parent, ‘BC8.3’, is the subject of U.S. Plant Pat. No. 25,507, filed Apr. 8, 2013, and issued May 5, 2015. The parent ‘8.1’ has attractive inflorescences but a growth habit that is not suitable for commercial production of ornamental potted plants. The plant ‘BC8.3’ has compact, attractive inflorescences with relatively large sepalous florets that ring and almost hide all of the non-sepalous florets in the center of the panicle, attractive sepal pigmentation, and stems that branch easily and are relatively strong.

The new variety ‘BCHY-11.026’ has relatively large sepalous florets, non-uniform sepal pigmentation at maturity and good commercial characteristics. ‘BCHY-11.026’ has compact, attractive inflorescences with relatively large sepalous florets that ring the non-sepalous florets in the center of the panicle. The color of the sepals changes as the plant ages. Below is a table comparing the new variety to similar varieties.

TABLE 1

| | | | | Commerical |
|---|---|---|---|---|
| | | | | variety ‘Venedig’ |
| | | U.S. | U.S. | which may be U.S. |
| | | Plant Pat. | Plant Pat. | Plant Pat. |
| | New Variety | 23,757 | 25,507 | 10,928 |
| | ‘BCHY-11.026’ | ‘BC6.1’ | ‘BC8.3’ | ‘Venice Raven’ |
| Leaf size | 8 cm wide × | 12 cm wide × | 10 cm × 16 cm | 11 cm wide x |
| | 10 cm long | 15 cm long | | 15.5 cm long-source |
| | | | | U.S. Plant Pat. |
| | | | | 10,928 |
| Plant height | 13″ in 4.5″ pot. | 15″ in 6″ pot. | 15″ in 6″ pot | 12″ in 6″ pot- |
| | | | | observed controls |
| | | | | grown alongside |
| | | | | ‘BC6.1’. |
| Stem strength | Strong | Stems are | Stems are | Strong-observed |
| | | strong but | relatively | controls grown |
| | | benefit from | strong. | alongside ‘BC6.1’. |
| | | being staked | | |
| Sepal | Both sides of | Upper side of | Upper side of | Upper side of |
| Pigmentation | sepals are | sepals is R.H.S. | sepals is R.H.S. | sepals is R.H.S. |
| | R.H.S. 68 C (red- | 86 A (violet | 73 A (red- | 84 A (violet |
| | purple group). | group); Under | purple group) | group). Under side |
| | | side of sepals is | in center, and | of sepals is R.H.S. |
| | | R.H.S. 88 D | margins of | 85 A (violet group) |
| | | (violet group) | sepals are | observed controls |
| | | | R.H.S. 56 A | grown alongside |
| | | | (red group). | ‘BC6.1’. |
| Sepalous Floret | 70 mm | 70 mm | 50 mm to | 70 mm-observed |
| Size—Diameter | | | 60 mm | controls grown |
| | | | | alongside ‘BC6.1’. |

The new cultivar ‘BCHY-11.026’ has been successfully asexually reproduced under controlled environmental conditions at a nursery in Half Moon Bay, Calif. under the direction of the inventor with its distinguishing characteristics remaining stable.

Asexual reproduction was first accomplished when vegetative cuttings were taken from the initially selected plant. Examination of asexually reproduced, successive generations grown in Half Moon Bay, Calif. show that the combination of characteristics as herein disclosed for ‘BCHY-11.026’ remains firmly fixed.

### DESCRIPTION OF THE DRAWINGS

The accompanying drawings consist of color photographs that show the typical plant form, including the inflorescence, foliage, and sepals.

FIG. 1 is a view of the entire plant showing its form, growth habit, dark green foliage, inflorescence, and the color of its sepals.

FIG. 2 is a top view of the entire plant showing its form, growth habit, dark green foliage, inflorescence, and the floor of its sepals.

FIG. 3 is a top view of an inflorescence.

FIG. 4 is a close-up view of the adaxial surface of a mature leaf.

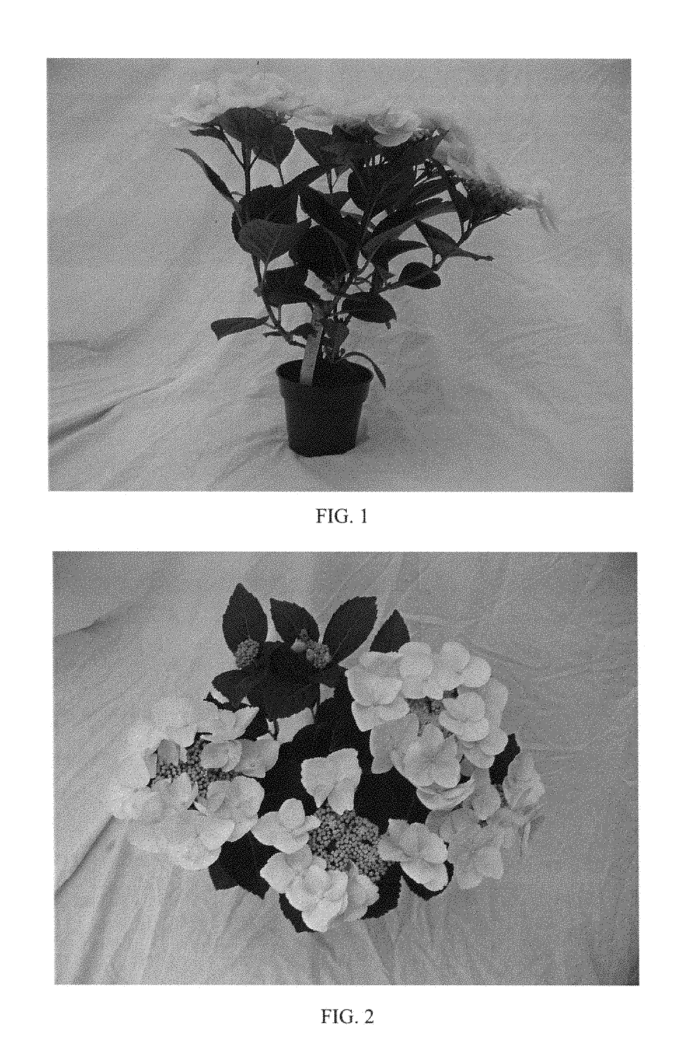

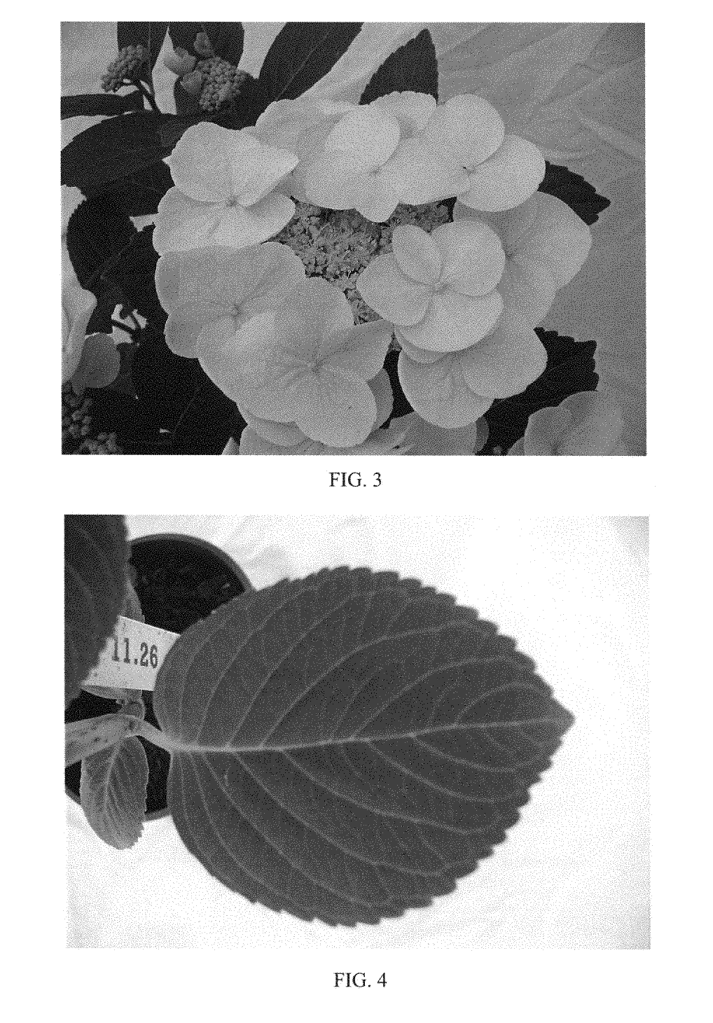

### DESCRIPTION OF THE NEW PLANT

The plant shown is approximately a year old. The plant started out as cuttings, taken from the stem of a grown plant. The plant was pinched early to promote lateral branches.

‘BCHY-11.026’ has not been observed under all possible environmental conditions. The phenotype may vary significantly with variations in environment such as temperature, light intensity, treatment with aluminum and day length. Color determinations were made with The Royal Horticultural Society (R.H.S.) Colour Chart, developed in association with the Flower Council of Holland, located in Lieden.
The plant:
   Origin.—Controlled cross. The new cultivar originated as a seedling from a controlled cross between the unpatented variety known as ‘8.1’ which was the seed parent and the commercial variety ‘13C8.3’ which was the pollen parent. ‘BC8.3’ is the subject of U.S. Plant Pat. No. 25,507.
   Form.—Upright, compact shrub. A typical plant with a mature inflorescence that is ready for sale is approximately 13″ high and has a diameter of 16″ when grown in a 4.5″ pot with appropriate soil amendments.
   Growth.—Upright, vigorous growth habit. Inflorescence is medium. The plant branches easily with shoots forming at the base of the plant. Lateral branches are similar in appearance and form to the main stems.
   Stems.—Lenticels are present. Lenticels are R.H.S. 86 A (violet group) and are 1 to 3 mm long. The surface of young stems is glabrous. Stems become woody as they age. The color of typical young stems and young lateral branches is R.H.S. 145 A (yellow-green group). The older portions of the stems are R.H.S. 199 A (grey-brown group). Younger portions of the stems are 5 mm in diameter. Older portions of the stems are 5-7 mm in diameter.
   Foliage.—Abundant. Leaves are opposite on stem and lateral branches. Shape of leaf. — Ovate with rounded base and acute apex. Margins are serrate. Texture. — Glabrous; veins dominate on the underside of the leaf and are sunken on the upper leaf surface. Color. — Mature leaves have an upper side that is R.H.S. 137 A (green group), and an under side that is R.H.S. 138 A (green group). Leaves are pinnately veined. The midvein and veins branching off the midvein are large and prominent on the underside of the leaves. Veins are R.H.S. 145 A (yellow-green group). Leaves are as wide as 8 cm and 10 cm long. Petioles are smooth and approximately 3 cm long and 4 mm wide. Petioles are R.H.S. 145 A (yellow-green group).
Buds:
   Form.—Globose with 4 to 5 connate, elliptic, smooth petals. Most buds, whether they will mature into sepalous or non-sepalous florets, have 5 petals. Buds in the center of the inflorescence are non-sepalous. The minority of buds will develop into sepalous florets. The buds are approximately 2 mm by 2 mm when very young. Buds can be 5 mm in diameter and still unopened. Color of mature buds is R.H.S. 73 C (red-purple group).
   Aspect.—Smooth.
   Arrangement.—Borne on branched panicles.
Inflorescence:
   Form.—Paniculate. Terminal. As many as 150 individual florets (both sepalous and non-sepalous) per inflorescence. Both sepalous florets and non-sepalous florets borne on the same panicle. Flowers do not produce a fragrance. The peduncle for the inflorescence is strong and upright. Many non-sepalous florets developing early on cymes that are later ringed by the sepalous florets that form at the outside of the panicle. Florets, both sepalous and non-sepalous, have anthers and style. Inflorescences are long-lasting.
   Size of inflorescence.—Compact and flat. Individual inflorescence size is dependent on the number of florets. A typical inflorescence can grow as large as 7″ in diameter, and 3″ high.
   Shape.—Clusters of numerous small florets; sepalous florets overlap one another. Sepals are persistent.
   Appearance.—not showy.
Florets:
   General.—The non-sepalous florets at the center of the inflorescence open first. Sepalous and non-sepalous florets are perfect and complete. Corolla: Generally, for both sepalous and non-sepalous florets there are 5 petals. Petals fall off easily of the non-sepalous florets. Petals are typically 4 mm long and 3 mm wide. Petals are R.H.S. 155 D (white group). Few lenticels are present on pedicels of both sepalous and non-sepalous florets, lenticels are no more than 1 mm long and very narrow. Lenticels are R.H.S. 59 B (red-purple group). Pedicel length for non-sepalous florets averages 4 mm. Pedicel length of sepalous florets is approximately 20 to 25 mm in length for plants of this age. Pedicels of both sepalous and non-sepalous florets continue to elongate as the inflorescence ages. Pedicels can be 73 D (red-purple group) to R.H.S. 145 C (yellow-green group) near floret when mature.
   Stamens.—8 to 10 stamens. Filament is approximately 2 to 4 mm long. Filament is R.H.S. 73 D (purple group). Anther is 1 mm long, regular and basally attached.
   Stigma.—Two to three style each, although most florets have two style. Each style has one stigma. Style is typically 1 mm long. Style is R.H.S. 73 D (red-purple group). Stigma is R.H.S. 73 D (purple group).
   Ovary.—Ovary is partially inferior.
   Sepalous florets.—General. — Veins dominate on the underside of the sepals. Number of sepals. — 4 to 5 sepals per floret, usually 4. Aspect of sepals. — Smooth and glaucescent. Shape of sepals. — Reniform with acuminate apex. Edges are entire, but with much crenation. Size of sepals. — Sepals at maturity are typically 40 mm long and 40 mm wide. Sepalous florets are typically 70 mm in diameter. The upper sides of the sepals are R.H.S. 68 C (red-purple group) and the undersides are the same. Pigmentation develops at the tips of the sepals and travels inward towards base of the sepals. Pigmentation lightens as the sepals reach maturity. Pigmentation of mature florets is 73 D (red-purple group).
   Fruit.—none.
   Disease and pest resistance.—unknown.

I claim:

1. A new and distinct Hydrangea macrophylla plant named ‘BCHY-11.026’ substantially as herein shown and described.
